(19) United States
(12) Patent Application Publication
(10) Pub. No.: US 2015/0297142 A1
(43) Pub. Date: Oct. 22, 2015

(54) DEVICE AND METHOD FOR EXTRACTING PHYSIOLOGICAL INFORMATION

(71) Applicant: KONINKLIJKE PHILIPS N.V., EINDHOVEN (NL)

(72) Inventor: Gerard De Jaam, Helmond (NL)

(21) Appl. No.: 14/417,800

(22) PCT Filed: Jul. 8, 2013
(86) PCT No.: PCT/IB2013/055576

§ 371 Date: Jan. 28, 2015

Related U.S. Application Data
(60) Provisional application No. 61/677,214, filed on Jul. 30, 2012.

(51) Int. Cl. A61B 5/00; A61B 5/024

(52) U.S. Cl. CPC A61B 5/7207; A61B 5/02433; A61B 5/7278; A61B 5/725; A61B 5/7485; A61B 2562/0233; A61B 2576/02

(57) ABSTRACT

The present invention relates to a device and method for extracting physiological information from electromagnetic radiation emitted or reflected by a subject (12). A data stream (30) derived from detected electromagnetic radiation is received, the data stream (30) comprising a first sequence (92; 152) of signal samples (94) indicative of various spectral portions. The data stream (30) is split into at least two deduced staggered sequences (96a, 96b, 96c) of registered signal samples (94a, 94b, 94c), wherein each of the deduced staggered sequences (96a, 96b, 96c) represents a defined spectral portion (82a, 82b, 82c) and comprises indicative signal samples (94a, 94b, 94c) spaced in time. Artificial samples (102a, 2b, 102c) are generated under consideration of proximate indicative signal samples (94a, 94b, 94c) so as to at least partially replace blank spaces (98) between the indicative signal 10 samples (94a, 94b, 94c), thereby generating a supplemented data stream (106). Preferably, a spectral composition of the signal samples (94a, 94b, 94c) in the first sequence (92; 152) is alternatingly influenced.

### FIELD OF THE INVENTION

The present invention relates to a device and a method for extracting physiological information from electromagnetic radiation emitted or reflected by a subject, wherein the physiological information is indicative of at least one at least partially periodic vital signal.

### BACKGROUND OF THE INVENTION

WO 2011/021128 A2 discloses a method and a system for image analysis including:

obtaining a sequence of images;

performing a vision based analysis of at least one of the sequence of images to obtain data for classifying a state of a subject represented in the images;

determining at least one value of a physiological parameter of a living being represented in at least one of the sequence of images, wherein the at least one value of the physiological parameter is determined through analysis of image data from the same sequence of images from which the at least one image on which the vision based analysis is performed is taken; and

classifying a state of the subject using the data obtained with the vision based analysis and the at least one value of the physiological parameter.

The document further discloses several refinements of the method and system. For instance, the use of remote photoplethysmographic (PPG) analysis is envisaged.

Basically, photophlethysmography is considered a conventional technique which can be used to detect blood volume changes which can be utilized to detect blood volume changes in the tissue of a monitored subject. Conventionally known PPG-approaches include so-called contact-PPG devices which can be attached to the skin of the subject, for instance to a finger tip. The PPG waveform typically comprises a pulsatile physiological waveform attributable to cardiac synchronous changes in the blood volume with every heart beat. Besides this, the PPG waveform can comprise further information attributable to respiration, oxygen saturation, and even further physiological phenomena.

Recently, so-called remote photophlethysmography has made enormous progress in that unobtrusive non-contact measurements have been demonstrated. Still, however, conventional PPG-approaches suffer from various draw-backs. Given that the recorded data such as captured reflected or emitted electromagnetic radiation (e.g., recorded image frames) always comprises, beside of the desired signal to be extracted therefrom, further signal components deriving from overall disturbances. Disturbances may occur, by way of example, from changing luminance conditions or movement of the observed subject. Furthermore, so-called specular reflection (basically “mirroring” incident radiation) is considered a huge challenge for remote-PPG approaches. Hence, a detailed precise extraction of the desired signals is still considered to pose major challenges for the processing of such data. There exists a general need for further improving the signal-to-noise ratio in remote PPG measurements.

A possible approach to this challenge may be directed to providing well-prepared and steady ambient conditions when capturing a signal of interest in which the desired signal component is embedded so as to minimize disturbing signal components overlaying the signal or interfering with the signal. However, such laboratory conditions cannot be transferred to everyday field applications as high efforts and preparation work would be required therefore. After all, vital signal detection is made even more difficult when amplitudes and/or nominal values of disturbing signal components are much larger than amplitudes and/or nominal values of desired signal components to be extracted. This applies in particular when facing considerable subject motion and poor illumination conditions. In the field of remote PPG, the magnitude of difference between the respective components (desired signals versus disturbing signals) can be expected to even comprise several orders.

### SUMMARY OF THE INVENTION

It is an object of the present invention to provide a system and a method for extracting physiological information from electromagnetic radiation emitted or reflected by a subject providing further refinements facilitating obtaining the desired signals with higher accuracy and preferably even under poor conditions, such as poor illumination and severe motion.

In a first aspect of the present invention a device for extracting physiological information from electromagnetic radiation emitted or reflected by a subject is presented, the device comprising:

an interface for receiving a data stream derived from detected electromagnetic radiation, the data stream comprising a first sequence of signal samples indicative of various spectral portions;

a data decomposer configured for splitting the data stream into at least two deduced staggered sequences of registered signal samples, wherein each of the deduced staggered sequences represents a defined spectral portion and comprises indicative signal samples spaced in time;

a data processor configured for generating artificial samples under consideration of proximate indicative signal samples so as to at least partially replace blank spaces between the indicative signal samples, thereby generating a supplemented data stream.

The present invention is based on the idea that accuracy and signal-to-noise ratio of the detected signals can be improved by splitting (or “unfolding”) the first sequence so as to obtain multiple “sub-sequences” providing a broader spectral basis for data processing. Consequently, a single sequence of signals can be split into at least two, preferably three sequences. Resulting blanks or gaps in the signal of each resulting split sequence can be filled or supplemented with artificial signals through interpolation. In this way, adverse side-effects of signal “chopping” (e.g., reduced temporal resolution, or, sample rate) can be prevented, at least to a certain extend. Signal decomposition or splitting can be performed at a “chopping” frequency. Consequently, in each of the resulting deduced staggered sequences less original signal samples than in the original sequence are present. In other words, the sample rate in each of the deduced staggered sequences is kept, even though less original samples are available. Blank gaps or spaces are filled with artificial samples. The artificial samples can be generated under consideration of neighboring original samples. Preferably, the artificial samples are motion-compensated.

It should be noted that replacing (or: filling) the blank spaces does not necessarily include entirely replacing each and every blank space between the indicative samples in the respective deduced staggered sequences. However, completely replacing the blank spaces is preferred. Still, replacing the blank spaces may also refer to filling at least one blank (of a blank space) between two indicative signal samples. For instance, when the data stream is split into three deduced staggered sequences of registered signal samples, a blank space may comprise two blank samples such that each staggered sequence is basically composed of recurring series of three samples (or: frames) comprising one indicative sample and a blank space formed by two blank samples. By way of example, the data processor can be configured for partially filling the blank spaces by replacing one of the two blank samples in the blank space.

As used herein, the defined spectral portion of each of the deduced staggered sequences can be formed by a defined wavelength interval the respective deduced staggered sequence represents. In other words, the device of the invention can make use of distinct portions of the electromagnetic spectrum (distinct channels) while it is not necessarily required that multi-channel capturing devices are utilized.

By way of example, the data stream received at the interface may comprise a series of signal samples (e.g., frames) covering recurring series of defined spectral portion compositions. Having knowledge of the composition of the data stream, the received first sequence can be split into the at least two deduced staggered sequences so as to broaden the data basis.

The device of the invention is particularly suited for being combined with low-cost sensor devices, in particular low-cost cameras. As used herein, the term low-cost camera may refer to a camera having a single sensor type having a limited spectral response behavior. For instance, low-cost IR-cameras can be used. Required IR-sensors may comprise a defined spectral sensitivity covering a single defined portion of IR-radiation. By contrast, commonly known (color) cameras may typically comprise three distinct sensor types (R, G, B). Consequently, signals at three wavelength intervals can be obtained. It is generally considered beneficial to capture the signals in at least two wavelength intervals (channels), since in this way adverse disturbances present in the signals can be accounted for. In other words, when combining the signals captured at three channels in an adequate way, two major distortion components (e.g., motion and specular reflection) can be dealt with.

The present invention “imitates” a data stream having more than just one (spectral) signal channel. Consequently, disturbance compensation measures can be applied as well, even though merely a single “channel” is used at the camera's end.

Furthermore, the device of the invention is particularly suited for processing an input data stream consisting of infrared (IR) signals. As indicated above, the first sequence can be split into at least two distinct staggered sequences which may represent distinct sub-intervals of the IR-interval of the electromagnetic spectrum. Hence, further application can be seen in environments which are barely illuminated. By way of example, overnight patient monitoring often involves little (visible) illumination, since bright illumination is considered to adversely affect the patient's sleep. Another particular field of application can be seen in workout monitoring in fitness centers or similar environments. In this connection, use is made of the fact that slightest changes in the reflection of incident radiation are also present and detectable in infrared radiation. The use of infrared signals may involve another benefit. Since the desired signals are embedded in slight fluctuations of the subject's skin's reflection of incident radiation which are caused by blood circulation (pulsation) in the subject's skin tissue, attention is given to the absorption behavior of the blood in the tissue and of the tissue itself (attributable to the type and amount of melanin in the subject's skin). In this context, it should be noted that especially for darker skins melanin absorption is huge and therefore remote PPG-measurements for this skin type can be considered a major challenge when facing poor illuminance conditions. Utilizing infrared radiation allows to profit from the fact that absorption of melanin is relatively low in this wavelength interval. Hence, a considerable amount of incident radiation may penetrate the skin and can be reflected by blood vessels so as to indicate blood pulsation-related volume changes thereof.

According to another aspect of the present invention the device further comprises a signal detector for extracting a continuous or discrete characteristic signal from the supplemented data stream, wherein the characteristic signal includes physiological information indicative of at least one at least partially periodic vital signal. The at least partially periodic vital signal can be selected from the group consisting of heart rate, heart beat, respiration rate, heart rate variability, Traube-Hering-Mayer waves, and oxygen saturation.

Since the supplemented data stream is basically composed of at least two, preferably three, signal channels, the desired signal can be extracted under consideration of algorithms allowing for further disturbance compensation measures. For instance, the extraction of the desired signal may comprise applying a linear combination of signals obtained from each of the at least two deduced sequences under consideration of defined coefficients. Furthermore, a temporal and/or spatial (i.e., local) normalization of each of the signals obtained at each of the at least two deduced sequences (channels) can be performed. However, in the alternative or in addition, further signal processing and optimization measures can be envisaged.

According to yet another aspect the device further comprises a sensor means, in particular a camera, configured for capturing electromagnetic radiation, wherein the sensor means comprises a defined response characteristic adapted to the at least one defined spectral distribution. As mentioned above, it is preferred to use a camera adapted to capture infrared radiation. In this connection, it is emphasized that a so-called “monochrome” low-cost camera can be utilized. It should be understood that the term “monochrome” does not necessarily refer to a spectral response characteristic of the camera providing an ideal single sensitivity peak at a singular wavelength. Instead, the term “monochrome” refers to a single sensor (type) in the camera basically having a single response curve in the radiation spectrum. Therefore, a “monochrome” IR-camera may cover a considerable portion of the infrared radiation interval. Still, it should be noted that such a camera usually comprises a single (color) channel. Nevertheless, according to the invention even a single-channel low-cost camera can be utilized, since the input data delivered via the single channel can be split into at least two deduced sequences (or: deduced channels) attributable to distinct portions of infrared radiation.

According to yet another aspect the device further comprises at least one source of electromagnetic radiation configured for directing radiation to the subject, in particular a source of infrared illumination. In this connection, it should be understood that both (or at least one of) the sensor means and the at least one source of electromagnetic radiation can be physically connected (fixed) to or even integrated into the device. However, each of or at least one of the sensor means and the at least one source of electromagnetic radiation also can be logically connected to the device in an alternative way. A physical connection may comprise a common housing or at least a physical attachment. A logical connection may comprise signal connections, either via cable or via wireless connections. Therefore, alternatively, the device of the invention can be regarded or interpreted as a system comprising distinct components cooperating and communicating in the desired way.

According to an even further preferred embodiment the device further comprises a signal scope expanding means for alternatively influencing a spectral composition of the signal samples in the first sequence. The signal scope expanding means can be utilized for enriching or preparing the first sequence of signal samples. The signal scope expanding means can influence the first sequence such that defined alternating samples or portions thereof are attributed to defined spectral portions or intervals. Having knowledge of the spectral composition of the signal samples allows for splitting the data stream into the at least two deduced sequences attributable to distinct channels, even when the initial data is captured via a low-cost “monochrome” sensor means. In this way, the received first sequence can be enhanced in terms of spectral information. The signal scope expanding means can be further configured for applying a periodically recurring spectral treatment (or: shift) to the signal samples.

According to another aspect the signal scope expanding means further comprises a clock-controlled filter means configured for selectively switching between at least two defined spectral response characteristics of the sensor means. Consequently, a given basic response characteristic of the sensor means can be alternatively influenced so as to capture electromagnetic radiation via a “monochrome” sensor means, while still preserving some spectral variety.

According to another aspect the signal scope expanding means comprises a clock-controlled filter means configured for selectively switching between at least two defined spectral distributions of radiation generated by the at least one source of electromagnetic radiation. So, alternatively, or in addition, also the radiation source can be influenced so as to selectively deliver radiation at alternating and periodically recurring spectral distributions. It should be noted that typically also a radiation source comprises a basic spectral distribution. By means of the filter, the distribution can be influenced so as to eventually capture reflected radiation via the “monochrome” sensor means still enabling to splitting the captured signal into at least two deduced sequences each of which attributed to a defined spectral portion. Consequently, it is preferred that an “influencing” frequency and a frame rate of the sensor means are somehow synchronized.

According to yet another embodiment the device (or system) comprises at least two illumination sources each of which configured for generating radiation of a distinct spectral composition, wherein the signal scope expanding means is further configured for time-sequentially alternatively driving the at least two illumination sources.

According to this aspect, the signal scope expanding means does not necessarily have to comprise a filter. By contrast, the signal scope expanding means can be implemented by a clock-controlled switch configured for switching between the at least two illumination sources so as to selectively direct radiation of a defined spectral composition to the subject of interest.

Hence, several embodiments of the signal scope expanding means may be envisaged. The signal scope can be expanded at the level of the illumination source or at the level of the sensor means. Furthermore, also a combination of a selectively influenced sensor means and at least one selectively influenced illumination source can be envisaged.

Still, however, also an embodiment of the device making use of the influenced sensor means but being implemented without an integrated source of electromagnetic radiation can be envisaged.

By way of example, the signal scope expanding means can be configured for cooperate with the sensor means and/or the at least one source of electromagnetic radiation such that the first sequence captured by the sensor means comprises samples alternatingly indicative of spectral portions having a wavelength peak at about 700 nm, about 800 nm, and about 900 nm. Hence, three deduced sequences can be extracted from the first sequence, even through merely a “monochrome” sensor means is used.

According to yet another aspect the data processor is further configured for generating interpolated artificial samples under consideration of proximate indicative signal samples such that the generated supplemented data stream comprises motion compensated artificial samples. In this way, the blanks or gaps remaining in each of the at least two deduced staggered sequences after splitting the first sequence can be filled such that the sample rate or frame rate can be maintained. Consequently, sets of single samples at the same time instant in the at least two deduced staggered sequences can be synchronized and processed accordingly.

According to another embodiment the device further comprises a skin segmentation means for detecting a region of interest in the subject, wherein the data processor is further configured for determining temporal displacement of the region of interest for generating the interpolated artificial samples.

It goes without saying, that skin portions of the subject of interest are considered highly indicative of the desired vital signals. Therefore, automatic skin segmentation and detection is considered beneficial. Furthermore, through skin segmentation subject motion can be detected and utilized for processing the interpolated artificial samples.

According to yet another embodiment the device further comprises a feature tracker for the detecting at least one distinct skin portion, in particular a face pattern, wherein a determined displacement of the at least one distinct skin portion is used for generating the interpolated artificial samples. Advantageously, skin segmentation and feature tracking can be combined so as to detect indicative regions and to track them during measurement and signal processing.

By way of example, motion compensation can make use of skin segmentation and/or feature tracking for detecting a center of (optical) gravity of the region of interest in the signal samples. Motion paths can be estimated upon tracking the center of (optical) gravity of the region of interest over time. Alternatively, the feature tracker can be embodied by a Lucas-Kanade tracker for estimating an optical flow so as to determine undesired motion between the subject and the sensor means.

According to another aspect of the device a frame rate of the sensor means and a splitting frequency of the data decomposer are synchronized, preferably the sensor means and the data decomposer are operated at an operating frequency amounting to twice as high as a mains frequency or to an integer fraction of a mains frequency of a power supply grid.

Upon a synchronizing the sensor means and the data decomposer, temporal alignment (synchronization) of sample capturing and spectral influencing can be ensured. It is therefore preferred to keep a whole number ratio between the frame rate of the sensor means and the splitting frequency of the data decomposer. Keeping a (frequency) distance from the mains frequency can avoid undesired beat frequencies which could adversely influence signal processing.

In a further aspect of the invention a method for extracting physiological information from electromagnetic radiation emitted or reflected by a subject is presented, the method comprising the steps of:

receiving a data stream derived from detected electromagnetic radiation, the data stream comprising a first sequence of signal samples indicative of various spectral portions;

splitting the data stream into at least two deduced staggered sequences of registered signal samples, wherein each of the deduced staggered sequences represents a defined spectral portion and comprises indicative signal samples spaced in time; and

generating artificial samples under consideration of proximate indicative signal samples so as to at least partially replace blank spaces between the indicative signal samples, thereby generating a supplemented data stream.

Advantageously, the method can be carried out utilizing the device for extracting information of the invention.

According to an embodiment the method further comprises the step of:

alternatingly influencing a spectral composition of the signal samples in the first sequence.

In yet another aspect of the present invention, there is provided a computer program which comprises program code means for causing a computer to perform the steps of the extracting method when said computer program is carried out on a computer.

As used herein, the term computer stands for a large variety of processing devices. In other words also mobile devices having a considerable computing capacity can be referred to as computing device, even though they provide less processing power resources than standard desktop computers. Furthermore, the term computer may also refer to a distributed computing device which may involve or make use of computing capacity provided in a cloud environment.

Preferred embodiments of the invention are defined in the dependent claims. It should be understood that the claimed methods and the claimed computer program can have similar preferred embodiments as the claimed device and as defined in the dependent device claims.

### BRIEF DESCRIPTION OF THE DRAWINGS

These and other aspects of the invention will be apparent from and elucidated with reference to the embodiments described hereinafter. In the following drawings:

FIG. 1 shows a schematic illustration of a general layout of a device in which the present invention can be used;

FIGS. 2a, 2b show diagrams representing spectral sensitivity graphs and spectral response characteristics;

FIGS. 3a, 3b and 3c show simplified schematic illustrations of a first sequence (FIG. 3a) which is split into deduced staggered sequences (FIG. 3b) which are eventually filled with artificial signal samples (FIG. 3c);

FIGS. 4a, 4b illustrate a sample signal frame and a series of signal frames to which motion compensation interpolation measures are applied;

FIG. 5 shows an exemplary frame having frame sections representing regions of interest in a subject;

FIG. 6 shows a schematic illustration of an exemplary signal scope expanding means;

FIG. 7 shows a schematic illustration of an alternative signal scope expanding means;

FIG. 8 shows a schematic illustration of yet another layout of an alternative signal scope expanding means; and

FIG. 9 shows an illustrative block diagram representing several steps of an embodiment of a method according to the invention.

### DETAILED DESCRIPTION OF THE INVENTION

The following section describes exemplary approaches to photophlethysmography, in particular remote photoplethysmography (remote PPG), utilizing several aspects of the device and method of the invention. It should be understood that single steps and features of the shown approaches can be extracted from the context of the respective overall approach. These steps and features can be therefore part of separate embodiments still covered by the scope of the invention.

Basic approaches to remote photoplethysmography are described in Verkruysse, W. at al. (2008), “Remote phlethysmographic imaging using ambient light” in Optics Express, Optical Society of America, Washington, D.C., USA, Vol. 16, No. 26, pages 21434-21445. WO 2011/042858 A1 discloses a further method and system addressing processing a signal including at least one component representative of a periodic phenomenon in a living being.

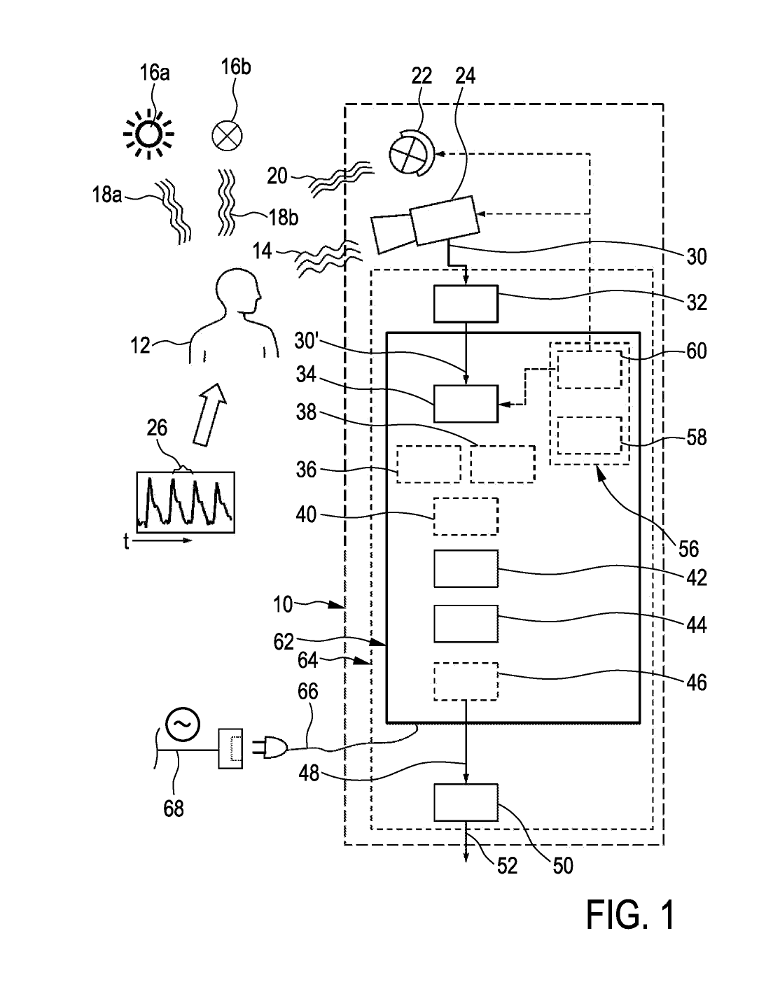

FIG. 1 shows a schematic illustration of a device for extracting physiological information which is denoted by a reference numeral 10. For instance, the device 10 can be utilized for recording image frames representing a remote subject 12 for remote PPG monitoring. The captured image frames can be derived from electromagnetic radiation 14 basically emitted or reflected by the subject 12. The subject 12 can be a human being or animal, or, in general, a living being. Furthermore, the subject 12 can be part of a human being highly indicative of a desired signal, e.g. a face portion, or, in general, a skin portion.

A source of radiation, such as sunlight 16a or an artificial radiation source 16b, also a combination of several radiation sources can affect the subject 12. It should be understood that the radiation sources 16a, 16b can be considered independent ambient radiation sources. Independent ambient radiation sources cannot be actively influenced by the device 10. The radiation sources 16a, 16b basically emit incident radiation 18a, 18b striking the subject 12. By contrast, the device 10 may also comprise at least one source of radiation 22 which can be selectively influenced or driven by the device 10. The at least one source of radiation 22 emits incident radiation 20 directed to the subject 12. The source of radiation 22 can be configured for delivering visible radiation or, more preferably, for delivering infrared (IR) or, even more preferred, near-infrared (NIR) radiation 20. The source of radiation 22 can be embodied by at least one light-emitting diode (LED) having a defined spectral characteristic. Furthermore, the source of radiation 22 can be embodied by an array or LEDs. For extracting information from the detected data, e.g., a sequence of image frames, a defined part or portion of the subject 12 can be recorded by a sensor means 24. The sensor means 24 can be embodied, by way of example, by a camera adapted for capturing information belonging to at least a spectral component of the electromagnetic radiation 14. Preferably, the sensor means 22 is embodied by an infrared (IR) or near-infrared (NIR) sensitive camera. Needless to say, the device 10 also can be adapted to process input signals, namely an input data stream, already recorded in advance and, in the meantime, stored or buffered.

As indicated above, the electromagnetic radiation 14 can contain a continuous or discrete characteristic signal which can be highly indicative of at least one at least partially periodic vital signal 26. The characteristic signal can be embedded in an (input) data stream 30. According to one embodiment, for data capturing, a potentially highly indicative portion of the subject 12 can be selected (or: preselected). The selection of the indicative portion may comprise masking the respective portion with a pixel pattern. When agglomerating respective signal pixel values of the pixel pattern at an instant (or: frame), a mean pixel value can be derived from the pixel pattern. In this way, the detected signals can be normalized and compensated for overall disturbances to some extent. The mean pixel value can be represented by a characteristic signal. The vital signal of interest 26 can be embedded in slight fluctuations (slight periodic property changes) of the characteristic signal. In the following, the captured data stream 30 can be considered a representation of a certain area of interest of the subject 12 which may cover an agglomerated pixel area covering a plurality of pixels. In FIG. 1, the vital signal 26 may allow several conclusions concerning heart rate, heart beat, heart rate variability, respiratory rate, or even oxygen saturation. Known methods for obtaining such vital signals may comprise tactile heart rate monitoring, electoral cardiography or pulse oximetry. To this end, however, obtrusive monitoring and measurement was required. As indicated above, an alternate approach is directed to unobtrusive remote measuring utilizing image processing methods.

The data stream 30 comprising the continuous or discrete characteristic signal can be delivered from the sensor means 24 to an interface 32. Needless to say, also a buffer means could be interposed between the sensor means 24 and the interface 32. Downstream of the interface 32 an input data stream 30′ can be delivered to a processing unit 62 indicated by a box. The processing unit 62 can be considered a computing device or, at least, part of a computing device driven by respective logic commands so as to provide for desired data processing. The processing unit 62 may comprise several components or units which are addressed in the following. It should be understood, that each component or unit of the processing device 62 can be implemented virtually or discretely. For instance, the processing unit 62 may comprise a number of processors, for instance, multi-core processors or single-core processors. At least one processor can be utilized by the processing unit 62. Each of the processors can be configured as a standard processor (e.g., central processing unit) or as a special purpose processor (e.g., graphics processor). Hence, the processing unit 62 can be suitably operated so as to distribute several tasks of data processing to adequate processors.

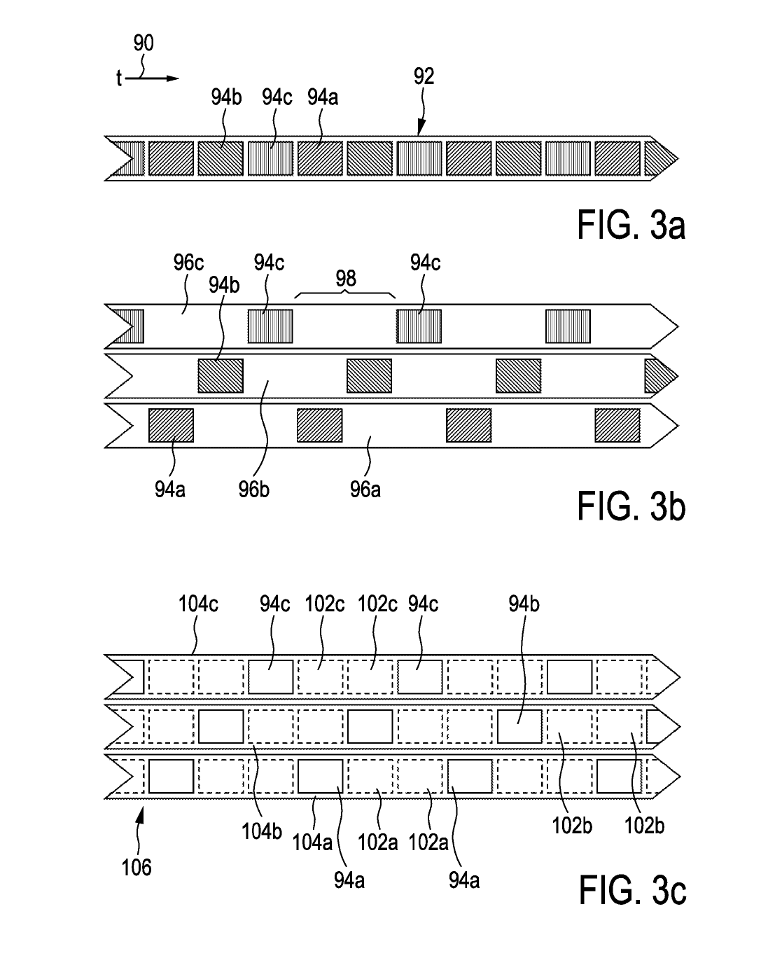

In accordance with one embodiment of the present invention, the processing unit 62 comprises a data decomposer 34 configured for splitting the input data stream (30, 30′) into deduced sub-sequences each of which representing a defined spectral portion. In this connection, reference is made to FIGS. 3a and 3b. Furthermore, a skin segmentation unit 36 and/or a feature tracker 38 can be provided. Both the skin segmentation unit 36 and the feature tracker 38 can be configured for pattern detection. Both components can be utilized for detecting a region of interest in the subject 12 which is considered highly indicative of the desired signal of interest. As indicated by dashed boxes, each of the components 36, 38 (as well as some of the components highlighted in the following) can be considered optional components further improving the data processing and extracting procedure.

In an alternative embodiment, pattern detection can be performed manually by a user of the device 10. For instance, the user can mask a face portion or skin portion of the subject 12 in a frame representing an initial frame for determining an initial frame section to be processed.

For instance, a filter 40 may be provided, in particular a frequency filter, preferably a low pass filter. The filter 40 can be configured for selectively filtering the input data stream 30, 30′ or, more preferably, the deduced sub-sequences derived by the data decomposer 34 from a first (singular) sequence embedded in the input data stream 30, 30′. The filter 40 can be configured for removing frequency portions of the input data which are clearly not related to assumed frequency behavior of the vital signal of interest. To some degree, frequency filtering can be considered a motion compensation measure, provided that motion related disturbances occur in the stop band of the filter.

It should be further noted that the term frequency, as often used herein, typically relates to (macroscopic) temporal frequencies occurring in processed data and signals. By contrast, when referring to the terms wavelength and spectrum, typically characteristics of electromagnetic radiation are addressed.

The processing unit 62 can further comprise a data processor 42 configured for generating artificial samples under consideration of proximate indicative signal samples so as to (re-)fill the deduced sub-sequences derived by the data decomposer 34. Further explanations in this regard are provided below in connection with FIGS. 3b and 3c. Still, the processing unit 62 can further comprise a signal detector 44 which is adapted to extract continuous or discrete characteristic signals from the data delivered thereto. Hence, the signal detector 44 can be configured for performing several signal extraction and enhancement algorithms. In particular, processed deduced sub-sequences derived from the first sequence can be utilized for that purpose. As mentioned above, the device 10 is configured for broadening a given signal basis in that a single first input sequence is split such that at least two deduced distinct sequences can be obtained. When transferring the single sequence (channel) data into multiple sequence (channel) data, several disturbances occurring in the captured data can be considered and reduced or even removed during subsequent processing.

Also a(n) (optional) data optimizer 46 can be provided in the processing unit 62. The data optimizer 46 can be configured for further enhancing a potentially indicative signal detected by the signal detector 44. For instance, also the data optimizer 46 can be embodied by a (frequency) filter. In the alternative, or in addition, the data optimizer 46 can be configured for applying a weighting algorithm or similar algorithms to the signals detected by the signal detector 44.

Downstream of the processing unit 62, an (output) interface 50 can be provided to which a processed data stream 48 can be delivered. Via the interface 50 output data 52 can be made available for further analyzes and/or for display measures.

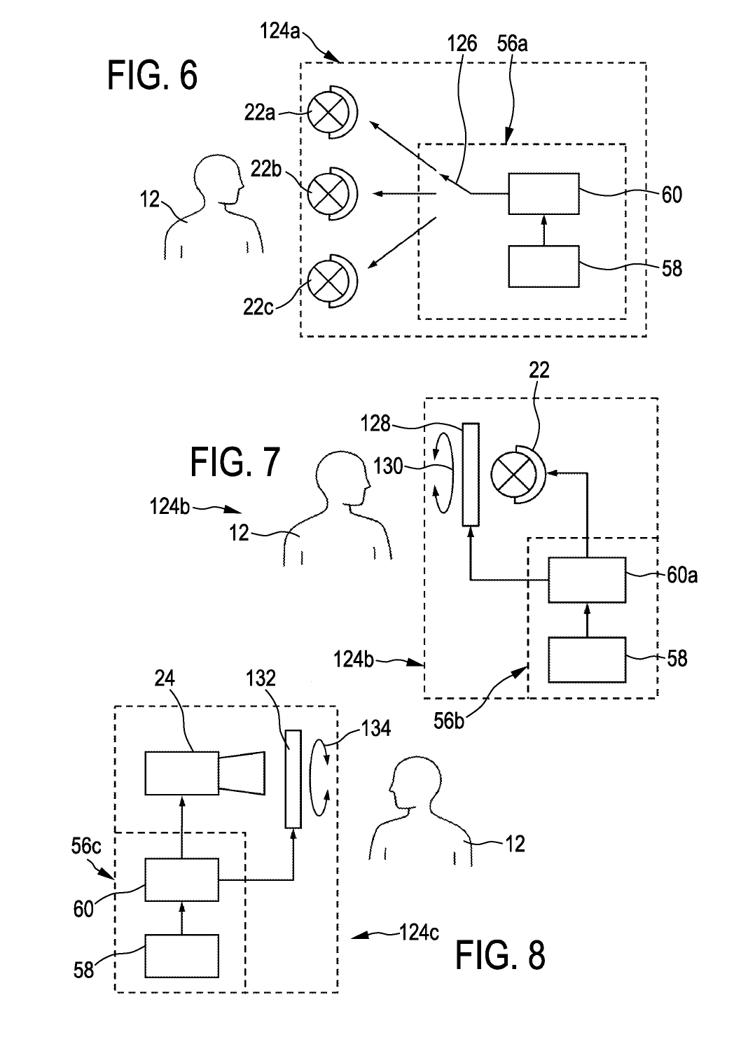

The processing unit 62 can further comprise a signal scope expanding means 56 which is configured for alternatingly influencing a spectral composition of the signal samples in the first sequence. Furthermore, embodiments of the signal scope expanding means 56 are addressed in connection with FIGS. 6, 7 and 8. The signal scope expanding means 56 can comprise a clock 58 which is configured for delivering a control or drive frequency which can be utilized for selectively controlling the spectral composition of the processed data. The clock 58 can be connected to a spectral controller 60. The spectral controller 60 can selectively influence the spectral data composition. The spectral controller 60 can alternatively or commonly control or drive the at least one source of radiation 22, the sensor means 24 and/or the data decomposer 34 so as to ensure that splitting of the first sequence of signal samples is synchronized with the alternating spectral composition of the captured data.

The processing unit 62 as well as the interfaces 32, 50 can be embodied in a common processing apparatus or housing 64. Reference numeral 64 can also describe a virtual system boundary. Still, also the sensor means 24 and the at least one source of radiation 22 can be integrated in the common processing housing 64. Conversely, it could be further envisaged to implement the device 10 as a distributed device. For instance, the sensor means 24 and the at least one source of radiation 22 can be positioned separate or distant from the processing unit 62. Moreover, functional entities of the processing unit 62 can be implemented in distributed processing devices which can be connected via cable or wireless networks.

The device 10 can be coupled via a connection line 66 to a power supply grid 68. The power supply grit 68 can be configured for delivering alternating current having an operation frequency, or, mains frequency. As indicated above, the mains frequency potentially can adversely affect the processing unit 62 by inducing so-called beat frequencies. It is therefore preferred to drive or operate the device 10 at a sample frequency (or: frame rate) which is sufficiently distant from the mains frequency.

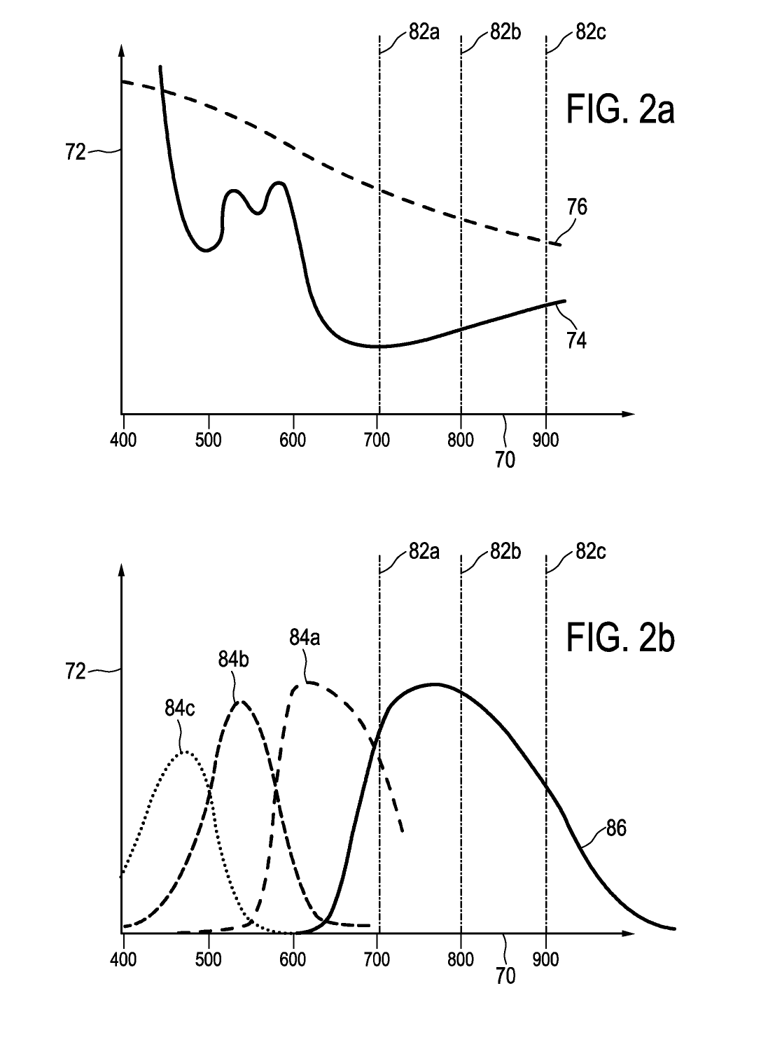

FIGS. 2a and 2b show spectral responsivity and/or absorption diagrams. An ordinate axis 70 represents electromagnetic properties, namely wavelength (scale marking in nanometers). The respective spectral interval indicated in FIGS. 2a and 2b covers visible radiation as well as a portion of short-wavelength ultraviolet (UV) radiation and a portion of long-wavelength infrared (IR) radiation. An axis of abscissas is denoted by a reference numeral 72. The axis 72 denotes dimensionless qualitative or proportional values indicating responsivity or absorption. In FIG. 2a two absorption graphs 74, 76 are illustrated. The absorption graph 74 describes typical blood absorption. The absorption graph 76 describes typical melanin absorption. When sensing or detecting a region of interest basically composed of skin, actual reflection may result from blood and melanin absorption (superimposed or overlayed by disturbances). The melanin absorption graph 76 basically decreases with increasing wavelength. The blood absorption graph 74 exhibits a characteristic curved form including humps. Blood absorption has a local minimum at about 680 nm. Furthermore, the blood absorption graph 74 comprises several local minima and maxima, in particular in the interval of radiation in which visible light is present. For determining the vital signal of interest which is related to actual blood perfusion, it is preferred to detect radiation at a wavelength interval in which neither the blood absorption nor the melanin absorption is too high. Therefore, the infrared region, preferably the so-called near infrared region, is well suited for the signal detection. By contrast, when utilizing standard cameras or sensor means, such as RGB-cameras, the interval of visible radiation needs to be addressed which is considered to be not an optimal choice in terms of blood and melanin absorption. For illustration purposes, FIG. 2b illustrates typical spectral sensitivity graphs 84a, 84b, 84c of a video camera having three kinds of (color) sensors or respective filters. Graph 84a may represent a red sensor or filter. Graph 84b may represent a green sensor or filter. Graph 84c may represent a blue sensor or filter. While conventional RGB-cameras are generally available at low costs, infrared cameras or sensor means having more than one kind of sensor type configured for detecting more than one wavelength interval in the infrared region are considered to be expensive and therefore not applicable for everyday applications.

On the other hand, low-cost infrared cameras are available covering a single wavelength portion in the infrared region. For instance, a spectral sensitivity graph 86 in FIG. 2b may represent a single-type sensor of an infrared camera.

As used herein, the term sensor typically refers to a certain type of kind of sensor arranged in an array in a digital sensor means. It goes without saying that a (digital) sensor means (e.g., a CCD camera) typically comprises a plurality of sensors. However, multi-channel cameras therefore require an array of multiple types of sensors while single-channel (monochrome) cameras merely require an array of a single sensor type.

As outlined above, for disturbance compensation measures, multi-channel signal recording is preferred. By combining the respective signals of each of the multiple channels, several disturbances (e.g., subject motion, specular reflections, and changes in ambient luminance) can be addressed. It is therefore desired to achieve a multi-channel or, at least, a quasi-multi-channel recording of signal samples in the desired wavelength interval, even though only one sensor type is provided in the camera. Coming back to FIG. 2b, it would be highly appreciated to selectively focus various spectral portions within the given spectral sensitivity graph 86 to define wavelength sub-intervals. A selection of such desired wavelength portions or segments is indicated by reference numerals 82a, 82b, 82c. Again, it is worth noting that the portions or segments 82a, 82b, 82c should not be understood or regarded in a limited way as “monochrome” segments in the strict sense of the term “monochrome”. Each of the reference numerals 82a, 82b, 82c may also stand for a wavelength interval.

A beneficial approach to the above issue is presented in connection with FIGS. 3a, 3b and 3c. FIG. 3a illustrates a series or first sequence 92 of consecutive signal samples 94 over time (refer to reference numeral 90 denoting a time axis). Each of the signal samples 94a, 94b, 94c may stand for a single frame in the first sequence 92. In the alternative, each of the signal samples 94 can also represent a plurality of consecutive frames. Preferably, each of the signal samples 94a, 94b, 94c covers the same period of time. Each of the signal samples 94a, 94b, 94c may represent a whole frame recorded by a sensor means. However, in the alternative, also a sub-section of a recorded frame may be represented by each of the signal samples 94a, 94b, 94c. The first sequence 92 can be referred to as a single-channel sequence of a (monochrome) sensor means. However, as indicated by defined hatchings, in the first sequence 92 an alternating series of various spectral portions can be present. For instance, signal samples 94a can be focused on section or segment 82a (refer to FIG. 2b). Accordingly, signal sample 94b can be indicative of radiation segment or section 82b. Furthermore, signal sample 94c can be highly indicative of radiation segment or section 82c. Consequently, even though only one signal channel is recorded, at least “quasi-multi-channel” information is embedded in the first sequence 92. Control of the actual spectral characteristic present in the respective signal sample 94 can be performed by the signal scope expanding means 56, refer to FIG. 1 and to FIGS. 6, 7 and 8 respectively.

FIG. 3b illustrates deduced staggered sequences 96a, 96b, 96c also referred to as sub-sequences. The deduced sequences 96a, 96b, 96c can be obtained through “splitting” the first sequence 92 under consideration of the defined alternating series of distinct spectral characteristics in the signal samples 94a, 94b, 94c. For instance, deduced sequence 96a initially can be composed of signal samples 94a highly indicative of spectral segment or portion 82a (FIG. 2b). Accordingly, deduced sequence 96b can comprise the signal samples 94b representative of the segment or portion of radiation 82b. Moreover, deduced sequence 96c can be composed of signal samples 94c representative of spectral segment or portion 82c. Consequently, spectral information embedded in the single-channel first sequence 92 can be unfolded and utilized during further processing.

In FIG. 3b, three deduced sequences 96a, 96b, 96c are derived from the initial first sequence 92. Consequently, each of the derived deduced sequences 96a, 96b, 96c comprises blank spaces or gaps 98, since merely each third signal sample 94 can be transferred from the initial first sequence 92 into a respective one of the deduced sequences 96a, 96b, 96c. In FIG. 3b each blank space 98 basically corresponds to the length of two signal samples 94. The blank spaces 98 are typically composed of at least one blank sample corresponding to an indicative sample (e.g., “synchronized” in terms of the sample rate). So in FIG. 3b each blank space 98 basically may comprise two blank samples.

FIG. 3c indicates that the blank spaces 98 (or at least one of their respective blank samples) in the deduced sequences 96a, 96b, 96c can be filled with so-called artificial signal samples 102a, 102b, 102c so as to re-establish more than one complete signal series resulting in at least two completed sequences 104a, 104b, 104c. Each of the artificial signal samples 102a, 102b, 102c may correspond to a single blank sample of the blank spaces 98. As mentioned above, each of the artificial signal samples 102a, 102b, 102c can be obtained through applying interpolation algorithms to neighbouring signal samples 94a, 94b, 94c. Neighbouring signal samples can comprise immediately or mediately preceding or succeeding signal samples of the same deduced sequence 96a, 96b, 96c. Preferably, sample interpolation is directed to motion compensation. Eventually, a supplemented data stream 106 can be obtained comprising the at least two completed sequences 104a, 104b, 104c in which each blank space is filled with respective artificial signal samples 102a, 102b, 102c.

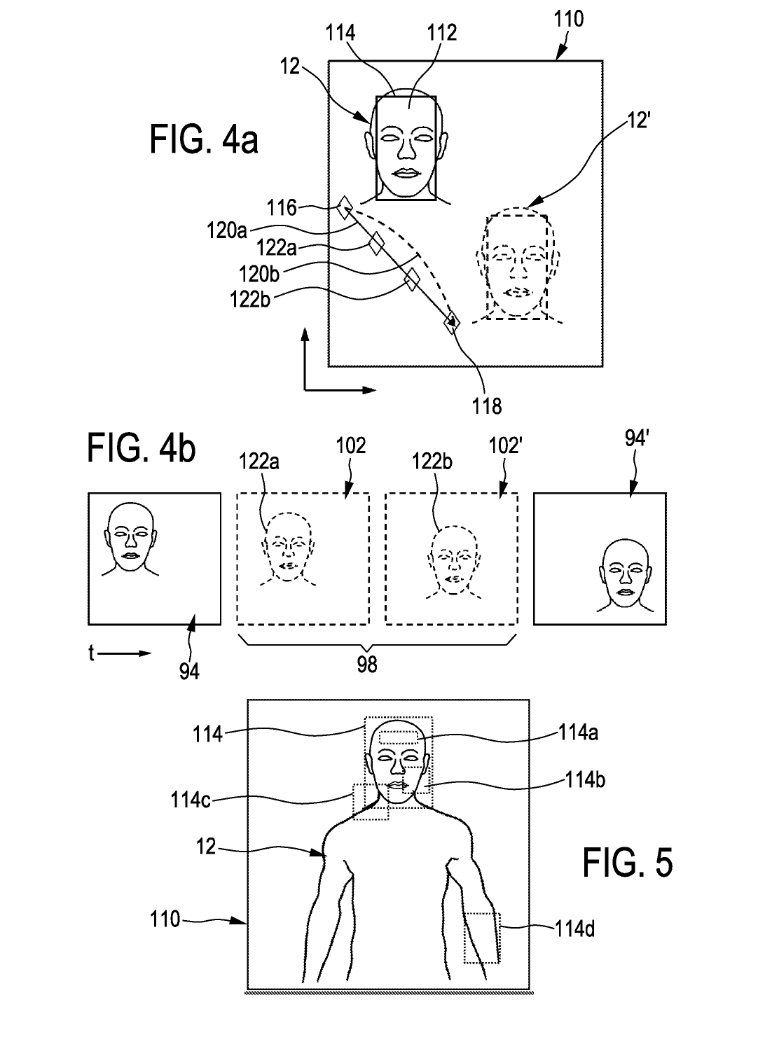

Signal sample interpolation can address motion compensation. In this connection, FIG. 4a shows a frame (or frame section) 110 representing a subject 12, in particular a face portion 112 of the subject 12. An exemplary region of interest 114 is indicated by a box. The region of interest 114 can be selected manually or utilizing the skin segmentation unit 36 and/or the feature tracker 38. Generally, pattern detection measures can be applied to a given representation of the subject 12 to be observed so as to automatically detect indicative portions which may serve as regions of interest 114. A shifted or displaced position of the subject 12 is indicated by reference numeral 12′. Subject 12′ may be represented in a succeeding signal sample 94′ (mediately) following a signal sample 94, refer to FIG. 4b. As shown in FIG. 3b, splitting the first sequence 92 basically leads to deduced sequences 96 comprising indicative signal samples 94 and gaps or blank spaces 98. Motion compensation can be directed to establish artificial signal samples 102, 102′ (FIG. 4b) filling or replacing respective blank spaces 98.

FIG. 4a further indicates displacement paths 120a, 120b which can be interpolated under consideration of determined respective initial positions 116 and tracked positions 118. The initial position 116 can represent the subject's 12 position in the signal sample 94 while the tracked position 118 may represent the subject's 12′ position in signal sample 94′. Basically, a straight path can be defined between the positions 116, 118 so as to determine a straight displacement path or vector 120a. However, the displacement path may also be curved, refer to the alternative displacement path 120b. Motion compensation through interpolation may be performed under consideration of more than one preceding or succeeding signal sample 94. Consequently, smooth motion transition can be assumed so as to determine curved displacement paths. Along each of the displacement paths 120a, 120b interpolated positions 122a, 122b can be determined. Based on the interpolated positions 122a, 122b, the artificial signal samples 102, 102′ can be established and included in each of the deduced sequences 96 so as to obtain the “refilled” supplemented sequences 102 forming the supplemented data stream 106 being composed of at least two channels.

FIG. 5 shows an (image) frame 110 exhibiting a representation of the subject 12. As mentioned above, several portions of the subject 12 to be monitored may serve as a region of interest 114 supposed to be highly indicative of the desired vital signals. For instance, a face portion as a whole can be represented in a region of interest 114. However, remote photophlethysmographic vital signal detection can also be applied to smaller regions of interest. For instance, a region of interest 114a can comprise a forehead portion of a face. An alternative region of interests 114b can comprise a cheek portion of a face. Furthermore, a region of interest 114c can comprise a neck portion. A further alternative region of interest 114d basically comprises a forearm portion of the subject 12 to be observed. Also a hand portion of the subject 12 can be observed as a region of interest.

FIGS. 6, 7 and 8 illustrate several embodiments of a signal scope expanding means 56 utilized for alternatingly influencing the spectral composition of the signal samples 94a, 94b, 94c in the first sequence 92. Basically, each of the signal scope expanding means 56 can be at least partially implemented in the processing unit 62 (FIG. 1).

FIG. 6 shows a signal scope expanding means 56a comprising a clock 58 and a spectral controller 60 as already outlined in connection with FIG. 1. Furthermore, the signal scope expanding means 56a comprises a driving switch 126 configured for selectively driving one of a plurality of sources of radiation 22a, 22b, 22c. The sources of radiation 22a, 22b, 22c can be embodied by respective LEDs or arrays of LEDs wherein each (type) of LEDs is adapted to a defined distinct wavelength portion. Altogether, the sources of radiation 22a, 22b, 22c and the signal scope expanding means 56a may form a signal enrichment unit 124a. The signal enrichment unit 124a can be synchronized with a frame rate of the sensor means or camera 24. Preferably, each of the sources of radiation 22a, 22b, 22c is configured for covering a defined distinct spectral segment or portion 82a, 82b, 82c (FIG. 2b) within a single spectral sensitivity or response characteristic 86 of a single-channel sensor means 24. Consequently, even though the sensor means 24 may comprise limited functionality and can be therefore purchased at low costs, “quasi-multi-channel” signal processing is achieved.

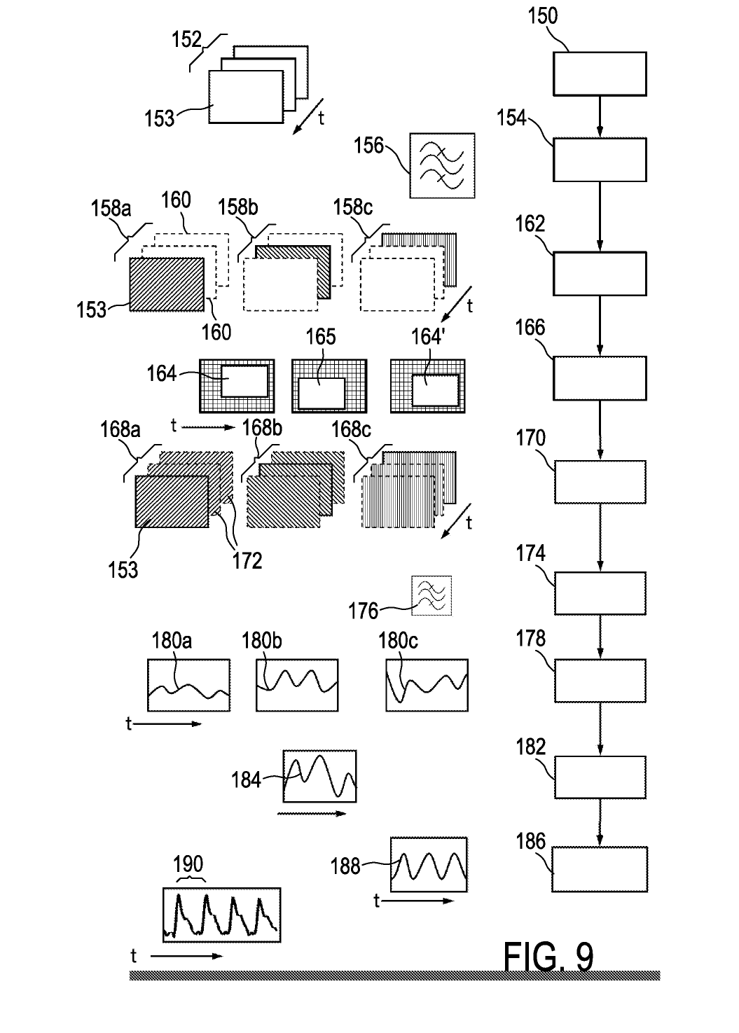

Basically the same functionality is provided by alternative signal enrichment units 124b, 124c presented in FIGS. 7 and 8, respectively. For instance, signal enrichment unit 124b comprises a signal scope expanding means 56b configured for controlling an illumination filter 128. The illumination filter 128 can be embodied by a movable filter array, refer to the double arrow 130. The illumination filter 128 can comprise portions of distinct defined filter characteristics. Driving the illumination filter 128 in accordance with a frame rate of the sensor means 24 enables capturing a time-sequential series of signal samples 94 covering defined alternating spectral portions 82a, 82b, 82c.

Signal enrichment unit 124c presented in FIG. 8 comprises a signal scope expanding means 56c which is configured for selectively operating a sensor filter 132 coupled to a sensor means 24. In this way, the recorded signals can be influenced in a desired manner at the level of the camera. Also the sensor filter 132 may comprise a moveable filter array having filter portions of defined distinct spectral sensitivity properties. Driving the sensor filter 132 (refer the double-arrow 134) may selectively influence the spectral response of the sensor means 24. Preferably, operation of the sensor filter 132 and frame rate of the sensor means 24 is synchronized. Further commonly known filters 128, 132, can be envisaged.

Each of the exemplary embodiments of the signal enrichment unit 124a, 124b, 124c can be implemented in the general layout of the device 10 shown in FIG. 1. These exemplary embodiments have in common that low-cost single-channel cameras may be utilized for signal detection, while multi-channel or, at least, “quasi-multi-channel” processing is enabled.

Having demonstrated several alternative exemplary approaches covered by the invention, FIG. 9 is referred to, schematically illustrating a method for extracting information from detected electromagnetic radiation.

Initially, in a step 150 an input data stream or a (first) input sequence 152 comprising several registered frames 153 is received. A time-axis is indicated by an arrow t. The data stream can be delivered from the sensor means 24, or from a data buffer or storage means. The data stream can be embodied, by way of example, by a sequence of image frames, or, image frame portions, varying over time. The image frames can comprise pixel data representative of infrared radiation. The input sequence 152 may comprise a representation of a subject of interest.

In a subsequent step 154 the input data stream comprising the sequence 152 is processed and split into at least two deduced sub-sequences 158a, 158b, 158c. Basically, signal splitting can be performed under consideration of a spectral filter 156 having at least two filter characteristics. In this way, the input sequence 152 can be registered according to various defined spectral portions already present in the sequence 152. The derived sub-sequences 158a, 158b, 158c may comprise indicative frames or samples 153 and blank spaces or gaps 160.

In another step 162 which may precede or succeed step 154, pattern detection is applied to the indicative samples 153 present in the data stream. In this way, regions of interest 164 can be determined.

In another step 166 motion interpolation is applied to the sub-sequences 158a, 158b, 158c. Motion compensation may comprise determining an initial position of the region of interest 164 and a resulting position of the region of interest 164′ in a signal sample immediately or mediately succeeding an initial signal sample. Consequently, mediate positions 165 of the region of interest can be determined. Eventually, artificial mediate signal samples 172 can be generated and utilized to fill or replace blank spaces 160 in the sub-sequences 158a, 158b, 158c so as to obtain filled or supplemented sequences 168a, 168b, 168c.

Subsequently, signal processing measures can be applied to the derived supplemented sequences 168a, 168b, 168c. It is worth noting that starting with an initial single-channel sequence 152, a multi-channel-representation of at least two supplemented or filled sequences 168a, 168b, 168c is obtained. Therefore, data processing step 170 can be directed to compensation for overall disturbances, such as subject motion and/or specular reflection; which may require a multi-channel-representation.

A filtering step 174 may follow which can address frequency filtering per channel or sequence. To this end, a frequency filter 176 may be utilized, which can be a low-pass filter or a bandwidth filter. To some degree, frequency signal filtering already can be considered a motion compensation measure. For instance low pass filtering may comprise a separation frequency at about 10 Hz.

In another step 178 signal collection measures can be performed. For instance, characteristic signal portions 180a, 180b, 180c may be obtained or derived from respective sequences 168a, 168b, 168c. For instance, signal agglomeration may be utilized agglomerating a plurality of pixel values in a region of interest so as to obtain a single representative value per frame.

In a signal derivation step 182 the characteristic signal portions 180a, 180b, 180c may be combined in a suitable manner, for instance by a linear combination. Eventually, a derived characteristic signal 184 can be obtained which already can be highly indicative of the desired vital signal. Still, in a step 186, further signal optimization measures can be applied to the derived characteristic signal 184. Consequently, an optimized signal 188 can be obtained allowing conclusions to be drawn about at least one at least partially periodic vital signal of interest 190. It should be understood that a time-based representation and/or a frequency-based representation of the signal of interest 190 might be of interest.

By way of example, the present invention can be applied in the field of health care, e.g., unobtrusive remote patient monitoring, general surveillances, security monitoring and so-called lifestyle environments, such as fitness equipment, or the like. Applications may include monitoring of oxygen saturation (pulse oximetry), heart rate, blood pressure, cardiac output, changes of blood perfusion, assessment of autonomic functions, and detection of peripheral vascular diseases. Needless to say, in an embodiment of the method in accordance with the invention, several of the steps described here can be carried out in changed order, or even concurrently. Further, some of the steps could be skipped as well without departing from the scope of the invention. This applies in particular to several alternative signal processing steps.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A device for extracting physiological information from electromagnetic radiation emitted or reflected by a subject, comprising:
an interface for receiving a data stream derived from detected electromagnetic radiation, the data stream comprising a first sequence of signal samples indicative of various spectral portions;
a data decomposer configured for splitting the first sequence into at least two deduced staggered sequences of registered signal samples, wherein each of the deduced staggered sequences represents a defined spectral portion and comprises indicative signal samples spaced in time; and
a data processor configured for generating artificial samples under consideration of proximate indicative signal samples so as to at least partially replace blank spaces between the indicative signal samples such that a supplemented data stream is generated.

2. The device as claimed in claim 1, further comprising a signal detector for extracting a continuous or discrete characteristic signal from the supplemented data stream, the characteristic signal including the physiological information indicative of at least one at least partially periodic vital signal.

3. The device as claimed in claim 1, further comprising a sensor configured for capturing electromagnetic radiation, wherein the sensor comprises a defined response characteristic adapted to at least one defined spectral distribution.

4. The device as claimed in claim 1, further comprising at least one source of electromagnetic radiation configured for directing radiation to the subject.

5. The device claimed in claim 1, further comprising a signal scope unit for alternatingly influencing a spectral composition of the signal samples in the first sequence.

6. The device as claimed in claim 5, wherein the signal scope unit comprises a clock-controlled filter configured for selectively switching between at least two defined spectral response characteristics of the sensor.

7. The device as claimed in claim 5, wherein the signal scope unit comprises a clock-controlled filter configured for selectively switching between at least two defined spectral distributions of radiation generated by the at least one source of electromagnetic radiation.

8. The device as claimed in claim 5, comprising at least two illumination sources, each of which configured for generating radiation of a distinct spectral composition, wherein the signal scope unit is further configured for time-sequentially alternatingly driving the at least two illumination sources.

9. The device as claimed in claim 1, wherein the data processor is further configured for generating interpolated artificial samples under consideration of proximate indicative signal samples such that the generated supplemented data stream comprises motion compensated artificial samples.

10. The device as claimed in claim 1, further comprising a skin segmentation unit for detecting a region of interest in the subject, wherein the data processor is further configured for determining temporal displacement of the region of interest for generating the interpolated artificial samples.

11. The device as claimed in claim 1, further comprising a feature tracker for detecting at least one distinct skin portion, wherein a determined displacement of the at least one distinct skin portion is used for generating the interpolated artificial samples.

12. The device as claimed in claim 1, wherein a frame rate of the sensor and a splitting frequency of the data decomposer are synchronized, such that the sensor and the data decomposer are at an operating frequency amounting to twice as high as a mains frequency or to an integer fraction of the mains frequency of a power supply grid.

13. A method for extracting physiological information from electromagnetic radiation emitted or reflected by a subject, comprising:
receiving a data stream derived from detected electromagnetic radiation, the data stream comprising a first sequence of signal samples indicative of various spectral portions;
splitting the first sequence into at least two deduced staggered sequences of registered signal samples, wherein each of the deduced staggered sequences represents a defined spectral portion and comprises indicative signal samples spaced in time; and
generating artificial samples under consideration of proximate indicative signal samples so as to at least partially replace blank spaces between the indicative signal samples such that a supplemented data stream is generated.

14. The method as claimed in claim 13, further comprising
alternatingly influencing a spectral composition of the signal samples in the first sequence.

15. (canceled)

16. A non-transient computer readable medium having instructions, when executed by a processor, for carrying out a method for extracting physiological information from electromagnetic radiation emitted or reflected by a subject, comprising:
receiving a data stream derived from detected electromagnetic radiation, the data stream comprising a first sequence of signal samples indicative of various spectral portions;
splitting the first sequence into at least two deduced staggered sequences of registered signal samples, wherein each of the deduced staggered sequences represents a defined spectral portion and comprises indicative signal samples spaced in time; and
generating artificial samples under consideration of proximate indicative signal samples so as to at least partially replace blank spaces between the indicative signal samples such that a supplemented data stream is generated.
